(19) United States
(12) Patent Application Publication
(10) Pub. No.: US 2023/0226160 A1
(43) Pub. Date: Jul. 20, 2023

(54) COMPOSITIONS AND METHODS FOR TREATING BACTERIAL INFECTIONS

(71) Applicant: MUCPHARM PTY LTD, Malvern, Victoria (AU)

(72) Inventors: David MORRIS, Kogarah, New South Wales (AU); Sarah VALLE, Kogarah, New South Wales (AU)

(21) Appl. No.: 18/007,134

(22) PCT Filed: Jul. 27, 2021
(86) PCT No.: PCT/AU2021/050820

§ 371 Date: Jan. 27, 2023

(30) Foreign Application Priority Data
Jul. 27, 2020 (AU) 2020902636

(51) Int. Cl. A61K 38/48; A61K 31/198; A61K 31/7036; A61K 31/43; A61K 38/46; A61P 31/04

(52) U.S. Cl. CPC A61K 38/4873; A61K 31/198; A61K 31/7036; A61K 31/43; A61K 38/465; A61P 31/04; C12Y 304/22033

(57) ABSTRACT

Disclosed herein is a composition comprising a biofilm degrading protease, a disulphide bond breaking agent and an antibiotic, as well as a method for debriding biofilm (e.g. on an implant) in a patient's body. The method comprises contacting the biofilm with a combination of a biofilm degrading protease and disulphide bond breaking agent.

### TECHNICAL FIELD

The present invention relates to compositions and methods for treating bacterial infections and, in some embodiments, to compositions and methods for debriding biofilm on implants in a patient's body.

### BACKGROUND ART

Bacterial infection is a common and serious complication of surgical procedures, and especially surgical procedures that involve the use of medical implants such as surgical mesh (e.g. for hernia repair), orthopaedic devices (e.g. joint replacements or metal plates for fracture fixation), vascular prosthesis, heart valves, as well as urinary and intravenous catheters. A major source of infection arises due to the formation of biofilm on the implant. Biofilm may also complicate the treatment of bacterial infections which occur because of other conditions. For example, secondary bacterial infections in cystic fibrosis, chronic obstructive pulmonary disease (COPD), severe asthma as well as other respiratory diseases can cause exacerbations of disease and are harder to treat if biofilm is present.

Biofilm is a complex extracellular matrix forming a home for microorganisms within which they are protected from systemic antibiotics due to limited penetration. The composition of biofilm depends on the bacteria present but generally includes extracellular polymeric substances, which are typically a polymeric conglomeration of extracellular polysaccharides, proteins, lipids and DNA. Furthermore, the bacteria can enter a senescent (sleeping) state in biofilm, even further protecting them from antibiotics. These factors make the bacteria contained in biofilm many orders of magnitude less sensitive to antibiotics.

Biofilm formation on implanted devices and prosthesis often leads to the need to remove the implant because antibiotics alone cannot prevent ongoing infection. For example, the use of mesh in surgical hernia repairs is proven to reduce recurrence rates and is currently used in around one million patients per year in the USA. However, infection can complicate up to about 10% of some procedures, and results in considerable morbidity for patients as well as a huge cost to the health system. To date, there has been little if any successful treatment of infected mesh other than by its removal, although such may be complicated (or simply impossible) due to tissue ingrowth and risk of morbidity. Surgical intervention to either remove the mesh (or a portion of the mesh) or clean the mesh is expensive, inconvenient and may not be entirely effective.

It would be advantageous if biofilm could be removed from implants and bacterial infections treated more reliably and without the need for surgical intervention.

### SUMMARY OF INVENTION

In a first aspect, the present invention provides a composition comprising a biofilm degrading protease, a disulphide bond breaking agent and an antibiotic.

As will be described in further detail below, the present inventor has discovered that the combination of therapeutically admissible amounts of a specific biofilm degrading protease and a disulphide bond breaking agent are surprisingly effective at degrading biofilm. The inventor believes that the results of their preliminary experiments lead to a reasonable prediction of the therapeutic applications disclosed herein. Further experiments, both currently underway and planned, should confirm the inventor's prediction.

In some embodiments, the biofilm degrading protease may be a cysteine protease. In some embodiments, the biofilm degrading protease may be selected from one or more of the group consisting of bromelain, papain, ficain, actinidain, zingibain, fastuosain and ananain. Advantages of using bromelain, in particular, will be described below.

In some embodiments, the disulphide bond breaking agent may be acetylcysteine.

In some embodiments, the antibiotic may be selected from one or more of the group consisting of: an aminoglycoside (e.g. gentamicin), a cephalosporin antibiotic and a penicillin antibiotic (e.g. ampicillin).

In some embodiments, the composition may further comprise an additional biofilm degrading agent. Such a biofilm degrading agent may, for example, be effective to degrade one or both of the DNA or PNAG (poly-N-acetylglucosamine) components of biofilm. In some embodiments, the biofilm degrading agent may selected from one or more of the group consisting of: DNase, calcium gluconate, dispersin B and subtilin.

In some embodiments, one or more additional therapeutic agents may be co-administered to the patient with the combination or composition. Such additional therapeutic agents may, for example be selected from one or more of the group consisting of: antiseptics and urea.

In a second aspect, the present invention provides a method for debriding biofilm on an implant (e.g. a permanent implant, such as a prosthetic, a surgical mesh, an orthopaedic device, a vascular prosthesis, a heart valve or an indwelling device such as an endotracheal tube, a central line, a urinary or intravenous catheter) in a patient's body, the method comprising contacting the biofilm with a combination of a biofilm degrading protease and a disulphide bond breaking agent.

The biofilm degrading protease and disulphide bond breaking agent may, in some embodiments, be administered simultaneously. Alternatively, in other embodiments, the biofilm degrading protease and acetylcysteine may be administered sequentially. Repeated administrations may be required for particularly tenacious biofilms.

In some embodiments, contacting the biofilm with the combination of the biofilm degrading protease and disulphide bond breaking agent comprises injecting the combination proximal to the implant (e.g. into a cavity or area surrounding the implant).

In some embodiments, the method may further comprise aspirating a resultant fluid from proximal to the implant (e.g. into a cavity or area surrounding the implant).

In some embodiments, the method may further comprise contacting the biofilm with an antibiotic. In some embodiments, the antibiotic may be administered simultaneously with or sequentially to the biofilm degrading protease and/or disulphide bond breaking agent. If administered sequentially, the antibiotic may be administered by the same route (e.g. by being administered into a cavity surrounding the implant or nebulised into the respiratory tract) or via a different route (e.g. by being administered systemically).

In some embodiments, the method may further comprise contacting the biofilm with an additional biofilm degrading agent. As noted above, such agents may be effective to degrade other components of the biofilm, such as DNA or PNAG.

In a third aspect, the present invention provides a method for treating a bacterial infection involving a biofilm in a patient (e.g. a secondary bacterial infection as a result of cystic fibrosis, COPD, severe asthma or other respiratory disease). The method comprises administering to the patient (e.g. by injection or by inhalation) a combination of a biofilm degrading protease, a disulphide bond breaking agent and an antibiotic. The antibiotic may again be administered simultaneously with or sequentially to the biofilm degrading protease and/or disulphide bond breaking agent.

In a fourth aspect, the present invention provides a method for treating a bacterial infection on an implant in a patient's body, the method comprising contacting the implant with a combination of a biofilm degrading protease, a disulphide bond breaking agent and an antibiotic.

In a fifth aspect, the present invention provides the use of a combination of a biofilm degrading protease and a disulphide bond breaking agent for debriding biofilm.

In a sixth aspect, the present invention provides the use of a combination of a biofilm degrading protease and a disulphide bond breaking agent for debriding biofilm on an implant in a patient's body.

In a seventh aspect, the present invention provides the use of a combination of a biofilm degrading protease, a disulphide bond breaking agent and an antibiotic for treating a bacterial infection.

In an eighth aspect, the present invention provides the use of a combination of a biofilm degrading protease, a disulphide bond breaking agent and an antibiotic for treating a bacterial infection on an implant in a patient's body.

In a ninth aspect, the present invention provides the use of a combination of a biofilm degrading protease and a disulphide bond breaking agent for the preparation of a medicament for debriding biofilm.

In a tenth aspect, the present invention provides the use of a combination of a biofilm degrading protease and a disulphide bond breaking agent for the preparation of a medicament for debriding biofilm on an implant in a patient's body.

In an eleventh aspect, the present invention provides the use of a combination of a biofilm degrading protease, a disulphide bond breaking agent and an antibiotic for the preparation of a medicament for treating a bacterial infection.

In a twelfth aspect, the present invention provides the use of a combination of a biofilm degrading protease, a disulphide bond breaking agent and an antibiotic for the preparation of a medicament for treating a bacterial infection on an implant in a patient's body.

In a thirteenth aspect, the present invention provides a composition comprising a biofilm degrading protease and a disulphide bond breaking agent for use in debriding biofilm.

In a fourteenth aspect, the present invention provides a composition comprising a biofilm degrading protease and a disulphide bond breaking agent for use in debriding biofilm on an implant in a patient's body.

In a fifteenth aspect, the present invention provides a composition comprising a biofilm degrading protease, a disulphide bond breaking agent and an antibiotic for use in the treatment of a bacterial infection.

In a sixteenth aspect, the present invention provides a composition comprising a biofilm degrading protease, a disulphide bond breaking agent and an antibiotic for use in the treatment of a bacterial infection on an implant in a patient's body.

Features of the methods of the third to sixteenth aspects of the present invention, and embodiments thereof, may be the same as those described herein in the context of the composition and method of the first and second aspects of the present invention, respectively. It is to be understood that any features and embodiments described herein in detail in relation to a specific aspect of the invention are equally applicable to other aspects of the invention. Other aspects, features and advantages of the present invention will be described below.

### BRIEF DESCRIPTION OF THE DRAWINGS

FIG. 1 is a graph showing the average absorbance values of biofilm after 48 hr treatment with bromelain (75 μg/ml) and a combination of bromelain (75 μg/ml)+acetylcysteine (1%, 0.5% and 0.25%) at 37° C.;

FIG. 2 is a graph showing the average absorbance values of biofilm after 48 hr treatment with bromelain (300 μg/ml) and a combination of bromelain (300 μg/ml)+acetylcysteine (1%, 0.5% and 0.25%) at 37° C.;

FIG. 3 is a graph showing the percentage of remaining 24-hour Staphylococcus aureus ATCC 6538 biofilms after a 4-hour treatment with N-Acetylcysteine (20 mg/ml)+Bromelain (5 μg/ml-100 μg/ml) in 5% glucose;

FIG. 4 is a graph showing the percentage of remaining 48-hour Staphylococcus aureus ATCC 6538 biofilms after a 4-hour treatment with N-Acetylcysteine (20 mg/ml)+Bromelain (5 μg/ml-100 μg/ml) in 5% glucose;

FIG. 5 is a graph showing the percentage of remaining 24-hour Staphylococcus aureus ATCC 6538 biofilms after a 4-hour treatment with N-Acetylcysteine (20 mg/ml)+Bromelain (25 μg/ml) and Gentamicin (12/16/35 μg/ml) in 5% glucose;

FIG. 6 is a graph showing the percentage of remaining 48-hour Pseudomonas aeruginosa ATCC 31461 biofilms after a 4-hour treatment with acetylcysteine (25/50 mg/ml) and bromelain (6.25-25 μg/ml) in 5% glucose;

FIG. 7 is a graph showing the percentage of remaining 48-hour Pseudomonas aeruginosa ATCC 31461 biofilms after a 4-hour treatment with acetylcysteine (10/15 mg/ml) and bromelain (12.5-40 μg/ml) in 5% glucose;

FIG. 8 is a graph showing the percentage of remaining 48-hour Pseudomonas aeruginosa ATCC 31461 biofilms after a 4-hour treatment with ampicillin, bromelain, and acetylcysteine in 5% glucose;

FIG. 9 is a graph showing the percentage of remaining 48-hour Pseudomonas aeruginosa PA01 398 biofilms after a 4-hour treatment with acetylcysteine (25/50 mg/ml) and bromelain (6.25-25 μg/ml) in 5% glucose;

FIG. 10 is a graph showing the percentage of remaining 48-hour Pseudomonas aeruginosa PA01 398 biofilms after a 4-hour treatment with gentamicin, bromelain, and acetylcysteine in 5% glucose;

FIG. 11 is a graph showing the percentage of remaining 48-hour Pseudomonas aeruginosa PA01 398 biofilms after a 4-hour treatment with ampicillin, bromelain, and acetylcysteine in 5% glucose;

FIG. 12 is a graph showing the percentage of remaining 48-hour Pseudomonas aeruginosa PA01 398 biofilms after a 4-hour combined treatment with ampicillin, bromelain, and acetylcysteine in 5% glucose;

FIG. 13 is a graph showing the percentage of remaining 48-hour Pseudomonas aeruginosa PA01 398 biofilms after a 4-hour staged treatment with bromelain and/or acetylcysteine in 5% glucose, washed then treated with ampicillin for 4-hours;

FIG. 14 is a graph showing the percentage of remaining 48-hour Pseudomonas aeruginosa PA01 398 biofilms after a 4-hour combined treatment with gentamicin, bromelain, and acetylcysteine in 5% glucose;

FIG. 15 is a graph showing the percentage of remaining 48-hour Pseudomonas aeruginosa PA01 398 biofilms after a 4-hour staged treatment with bromelain and/or acetylcysteine in 5% glucose, washed then treated with gentamicin for 4-hours;

FIG. 16 is a graph showing the percentage of remaining Staphylococcus aureus ATTC 6538 biofilm after 4-hrs treatment with DNase (25-200 μg/ml), with and without bromelain and acetylcysteine (200 μg/ml/3.125 mg/ml); and

FIG. 17 is a graph showing the percentage of remaining Staphylococcus aureus ATTC 6538 biofilm after 4-hrs treatment with DNase (25-200 μg/ml), with and without bromelain and acetylcysteine (100 μg/ml/6.25 mg/ml).

### DETAILED DESCRIPTION OF THE INVENTION

As noted above, in its broadest form the present invention provides a composition comprising a biofilm degrading protease (e.g. bromelain), a disulphide bond breaking agent (e.g. acetylcysteine) and an antibiotic (e.g. gentamicin or ampicillin). Further agents may be included in the composition in order to even further improve its beneficial effects, as will be described below.

The present invention also provides a number of therapeutic methods. In a method for debriding biofilm on an implant in a patient's body, the biofilm is contacted with a combination of a biofilm degrading protease and a disulphide bond breaking agent. In a method for treating a bacterial infection on an implant in a patient's body, the implant is contacted with a combination of a biofilm degrading protease, a disulphide bond breaking agent and an antibiotic. In a method for treating a bacterial infection involving biofilm in a patient, a combination of a biofilm degrading protease, a disulphide bond breaking agent and an antibiotic are administered (e.g. via injection or inhalation) to the patient.

The present invention has been made following the inventor's discovery that bromelain is capable of debriding biofilm on surgical mesh implanted in a patient. The amount of bromelain required to achieve this effect was, however, an amount at which the inventor had expected would cause adverse issues known to be associated with systemic administration of bromelain, such as its fibrinolytic action and effect on bleeding. Surprisingly these issues were not observed in the patient that was treated, but the inventor would not expect the same result for all patients. The risks of administering relatively large quantities of bromelain are well known, and the inventor expects that an intraperitoneal injection of bromelain at a dose of greater than about 100 μg/ml would carry a significant risk of an adverse event.

Subsequent experiments by the inventor have surprisingly found that, despite acetylcysteine (also referred to herein as N-acetylcysteine or NAC) itself having no appreciable effect on biofilm itself (at therapeutically appropriate dosages), a synergistic effect is observed and biofilm is degraded when a combination of relatively low (and likely tolerable) amounts of bromelain and acetylcysteine are brought into contact with the biofilm. Indeed, amounts of bromelain as low as 5 μg/ml bromelain in combination with acetylcysteine (20 mg/ml) have been demonstrated to act synergistically and degrade biofilm. The inventors cannot presently explain why this observed synergy occurs, but note it is unlikely that NAC's disulphide bridge bond breaking functionality is the primary reason, given that it appears to be ineffective of itself.

As noted above, and as will be described in further detail below, the inventor has discovered that the combination of therapeutically tolerable amounts of a specific biofilm degrading protease and acetylcysteine are surprisingly effective at degrading biofilm. The inventor believes that the results of their preliminary experiments support a reasonable prediction of the therapeutic applications disclosed herein. The inventor believes that the results of their preliminary experiments also support a reasonable prediction that other biofilm degrading proteases will have utility in the present invention. Further experiments, both currently underway and planned, should confirm the inventor's prediction.

The present invention may be used to treat bacterial infections involving biofilm in any suitable patient or subject. In some embodiments, the patient is a mammalian subject. Typically, the patient will be a human patient, although other subjects may benefit from the present invention. For example, the subject may be a pig, mouse, rat, dog, cat, cow, sheep, horse or any other mammal of social, economic or research importance.

The present invention may be used to debride biofilm on any substrate, natural or introduced. One particular application of the invention is for debriding biofilm on an implant in a patient's body. Such implants may include permanent implants, such as prosthetic implants, surgical mesh, orthopaedic devices (e.g. prosthetic knees or hips), vascular prosthesis and heart valves, for example. Indwelling devices such as endotracheal tubes, central lines and urinary or intravenous catheters may also attract biofilm development in a patient. Indeed, the inventor notes that biofilm on endotracheal tubes causes ventilator associated pneumonia, which may be prevented by the present invention. As would be appreciated, furthermore, many other implants are routinely used in surgery, and the applicability of the present invention extends to all such implants.

Another application of the invention is for treating infections where biofilm has formed in a patient (e.g. in their lungs or airways), for example as the result of a secondary bacterial infection as a result of cystic fibrosis, COPD, severe asthma or other respiratory diseases. In such applications, administration of the biofilm degrading protease and a disulphide bond breaking agent in accordance with the present invention may increase the efficacy of the antibiotic.

The present invention involves the use of a combination of a biofilm degrading protease and a disulphide bond breaking agent, each of which will be described in turn below.

### Biofilm Degrading Protease

A biofilm degrading protease is proteolytic enzyme which degrades biofilm. As used herein, the term “degrading biofilm”, and the like, is to be understood to encompass any mechanism which results in the biofilm being dissolved, dispersed, liquified, digested or otherwise broken down such that the biofilm can be removed from the substrate upon which it had formed.

Given the preliminary experimental data for bromelain, which is a protease enzyme that has been observed by the inventor to dissolve biofilm and debride it from a surgical mesh implanted in a patient, the inventor believes that any biofilm degrading protease may have applicability in the present invention, with routine trial and experimentation being all that would be required (in light of the teachings contained herein) in order to determine any particular protease's suitability.

The biofilm degrading protease may, for example, be a cysteine protease. Cysteine proteases (also known as thiol proteases) degrade proteins via a common catalytic mechanism, and are commonly sourced from fruits including the papaya, pineapple, fig and kiwifruit. Examples of potentially suitable cysteine proteases include bromelain and papain (extracted from papaya).

There are other plant-derived proteolytic enzymes that express the same characteristics as Bromelain and the inventors expect that plant-derived protease enzymes may produce a similar effect. Again, routine experimentation should be able to confirm the suitability of any particular plant-derived protease enzyme. In some embodiments, for example, the plant-derived protease enzymes may be selected from one or more of the group consisting of Bromelain and Ananain (extracted from pineapple), Papain (extracted from papaya), Ficain (extracted from figs), Actinidain (extracted from fruits including kiwifruit, pineapple, mango, banana and papaya), Zingibain (extracted from ginger) and Fastuosain (a cysteine proteinase from Bromelia fastuosa). Asparagus, mango and other kiwi fruit and papaya proteases may also be used.

As used herein, Bromelain is to be understood to encompass one or more of the biofilm affecting and, optionally, otherwise therapeutically active substances present in the extract of the pineapple plant (Ananas Comosus). Bromelain is a mixture of substances (including different thiol endopeptidases and other components such as phosphatase, glucosidase, peroxidase, cellulase, esterase, and several protease inhibitors) and it may not be necessary for all of these substances to be included in the combination, provided that the fraction of the substances in the combination can at least affect the biofilm in a manner that results in it degrading. The Bromelain used in the experiments described herein was commercially sourced from Enzybel Group.

### Disulphide Bond Breaking Agent

The present invention also includes a disulphide bond breaking agent. In the proof of concept experiments conducted by the inventor, acetylcysteine (NAC) was used. Acetylcysteine is an antioxidant with reducing potential in biological systems, and is known to cleave disulphide bridges in proteins. As the integrity of many proteins are dependent on disulphide bridges, the inventor postulates that their breakage by acetylcysteine will cause unfolding of proteins in the biofilm, which may help to degrade the biofilm. As noted above, however, given that acetylcysteine is relatively ineffective on its own, other mechanisms are likely to be contributing to the effect disclosed herein.

Advantageously, acetylcysteine is an approved product for paracetamol overdose where 21 g is given systemically over a 24-hour period. Acetylcysteine is also used as a mucolytic as a treatment for cystic fibrosis and chronic obstructive pulmonary disease, which is administered via inhalation, either 10% or 20% in 4 ml up to four times daily. Thus, regulatory approvals for medicaments including acetylcysteine may be easier to obtain.

Embodiments of the present invention as including acetylcysteine are described herein. A person skilled in the art would, however, appreciate that the teachings contained herein could likely be adapted, using routine trials and experiments, for any agent having a similar effect. Other disulphide bond breaking agents which the inventors believe may be used in the present invention include cysteamine, endosteine, s-carboxymethylcysteine.

The relative proportions of the biofilm degrading protease and acetylcysteine in the combination may vary between about 5 μg/mL and about 200 μg/mL of the biofilm degrading protease and between 1% to 10% (w/v) of the acetylcysteine. Amounts of proteolytic enzyme much lower than 5 μg/mL may not be effective and amounts higher than 200 μg/mL would be more likely to cause undesirable side effects. In some embodiments for example, about 5 μg/mL, 10 μg/mL, 15 μg/mL, 20 μg/mL, 30 μg/mL, 40 μg/mL, 50 μg/mL, 60 μg/mL, 80 μg/mL, 100 μg/mL, 150 μg/mL, 200 μg/mL of the biofilm degrading protease may be present. In some embodiments for example, about 1%, 2%, 3%, 4%, 4%, 6%, 7%, 8%, 9% or 10% (w/v) of the acetylcysteine may be present.

It is envisaged that, for some biofilms, repeated treatments may be necessary in order to completely eradicate the biofilm.

The combinations and compositions of the present invention may also include an antibiotic. Any antibiotic that will provide a therapeutic effect in the context of the present invention (i.e. any antimicrobial that is effective against biofilm forming bacteria) may be used. Antibiotics which the inventor expects will be useful include aminoglycosides (e.g. gentamicin), cephalosporins, fluoroquinolones, macrolides and penicillin antibiotics (e.g. ampicillin).

The combination or composition of the biofilm degrading protease, disulphide bond breaking agent and, optionally, antibiotic may be administered to the patient in any manner that provides the intended therapeutic effect. Typically, in therapeutic applications where biofilm is to be debrided from an implant, the composition would be adapted for injection into a patient, at or proximal to the biofilm. The phrase “proximal” to the biofilm is to be understood as meaning that the composition is administered whereby it can make contact with the biofilm and react to produce the effects described herein.

In therapeutic applications where biofilm is located in the lungs or airway of the patient, the composition may be adapted for inhalation by the patient. In some embodiments, for example, the composition may be nebulised in order for a patient to inhale. In this manner, bacterial infections involving biofilm, such as those described herein, can be treated.

Acetylcysteine is known to interfere with the activity of some antibiotics and the inventor's preliminary experiments indicate that combination treatments may not be as effective as sequential treatments in increasing biofilm destruction. Indeed, the inventor's preliminary data indicates that an initial treatment with bromelain/acetylcysteine is effective to break down the biofilm matrix, with subsequent treatment with the antibiotic effectively targeting the more exposed bacteria. The timing experiments described below makes it seem likely that repeat treatment every 6-24 hours for multiple days, with systemic or targeted delivery (e.g. respiratory, cavity, joint, etc) of the antibiotic every 24 hours might be clinically relevant.

In combinations and compositions including an antibiotic, the antibiotic may be present in the composition in any amount that produces an antibacterial effect. For example, concentrations of between about 10 μg/mL and about 100 μg/mL of gentamicin have been found by the inventor to have an antibacterial effect in the context of the present invention. Similarly, concentrations of between about 25 μg/mL and about 200 μg/mL of ampicillin have been found to have an antibacterial effect. It is within the ability of a person skilled in the art to determine an appropriate dose and class of antibiotic, taking factors such as the location of the implant, extent of the biofilm and types of bacteria present, for any given patient. In some embodiments, therefore, the antibiotic may be present in amount of about 10 μg/mL, 20 μg/mL, 30 μg/mL, 40 m/mL, 50 μg/mL, 60 μg/mL, 70 m/mL, 80 m/mL, 90 μg/mL, 100 m/mL, 110 μg/mL, 120 m/mL, 130 μg/mL, 140 m/mL, 150 μg/mL, 160 m/mL, 170 μg/mL, 180 μg/mL, 190 μg/mL or 200 m/mL.

The combinations and compositions of the present invention may also include an additional biofilm degrading agent (i.e. in addition to the biofilm degrading protease). Any agent that will degrade biofilm when used in accordance with the present invention, as described herein, and which does not deleteriously affect the performance of the invention may be used.

In some embodiments, the biofilm degrading agent may be an agent that functions to degrade biofilm in a different manner to that of the biofilm degrading protease. In some embodiments, the biofilm degrading agent may act to further enhance the degrading effect of the biofilm degrading protease, or may be effective against other structural or functional components of the biofilm. As noted above, biofilm includes extracellular polymeric substances including extracellular polysaccharides, proteins, lipids and DNA. The biofilm degrading agent may therefore, for example, degrade either or both of the DNA or poly-N-acetylglucosamine (PNAG) components of biofilm.

Poly-N-acetylglucosamine (PNAG), for example, is an extracellular polysaccharide that forms a significant part of staphylococcal biofilms and the inventor expects that an even further enhanced effect will occur if PNAG-degrading agents such as calcium gluconate, dispersin B and subtilin are included in the present invention. Experiments to confirm this expectation are presently underway.

DNA is also a significant component of biofilm, and the inventor has demonstrated (described in further detail below) an even further enhanced effect when DNA-degrading agents such as DNase are included in the present invention.

In compositions including an additional biofilm degrading agent, the agent may be present in the composition in any amount that produces a beneficial effect. For example, in the case of DNase, an amount of from about 5 to 200 μg/mL would be expected to provide the beneficial effects described herein. In some embodiments, the additional biofilm degrading agent may be present in amount of about 5 μg/mL, 10 μg/mL, 20 μg/mL, 30 μg/mL, 40 m/mL, 50 ng/mL, 60 μg/mL, 70 ng/mL, 80 ng/mL, 90 ng/mL, 100 ng/mL, 110 ng/mL, 120 ng/mL, 130 m/mL, 140 μg/mL, 150 m/mL, 160 μg/mL, 170 m/mL, 180 μg/mL, 190 m/mL or 200 μg/mL. It is within the ability of a person skilled in the art to determine an appropriate quantity of any such additional biofilm degrading agent. In some embodiments, two or more additional biofilm degrading agent may provide beneficial effects, especially if they act on different components of the biofilm.

Any additional therapeutic agent having an appropriate indication in the context of treating a bacterial infection may also be co-administered to the patient. In some embodiments, the co-administered therapeutic agent may provide symptomatic relief and not a specific antibacterial effect. Examples of additional therapeutic agents include antiseptic agents and urea (which may extract non-covalently bound extracellular matrix proteins).

When needed (or beneficial), the quantities of such additional therapeutic agents may be determined on an as-needed basis using no more than routine trials and experimentation.

As noted above, the present invention provides a method for debriding biofilm on an implant in a patient's body. The method comprises contacting the biofilm with a combination of a biofilm degrading protease and a disulphide bond breaking agent. Biofilm degrading proteases and disulphide bond breaking agents suitable for use in this method of the present invention include those described above.

The combination of the biofilm degrading protease and disulphide bond breaking agent may be caused to contact the biofilm using any suitable technique, depending mainly on the location of the biofilm/implant in the patient's body. Typically, the combination of the biofilm degrading protease and disulphide bond breaking agent would be injected into the patient proximal to the implant, this being a non-surgical procedure which would be simpler and less risky to perform. In serious cases, however, a radiologically placed drain (e.g. pigtail catheter) or surgical incision could be made in order for the implant/biofilm to be more fully accessed.

Typically, the method would further comprise aspirating a resultant fluid from proximal to the implant. In this manner, degraded biofilm etc. would be removed from the body before it could spread, potentially causing secondary infections. Alternatively, however, any such fluid might be allowed to drain using a surgical drain.

The biofilm degrading protease and disulphide bond breaking agent may be administered in any manner that results in biofilm debridement. They may, for example, be simultaneously administered (e.g. in a single composition) or sequentially administered (e.g. in separate compositions, one after the other).

In some embodiments, the method may further comprise contacting the biofilm with an antibiotic, where its antibacterial properties provide a beneficial effect (e.g. killing any bacteria that were entrained in the biofilm but which are now exposed). In such embodiments, the antibiotic may be administered simultaneously with or sequentially to the biofilm degrading protease and/or acetylcysteine. Antibiotics suitable for use in the method of the present invention include those described above.

Another surgical procedure which the inventor expects the present invention will be applicable is DAIR (debridement, antibiotics and implant retention) surgery. DAIR surgery is performed in the event of periprosthetic joint infection (PJI), a complication associated with hip and knee arthroplasty, and involves surgically exposing the implant, followed by debriding and washing the implant's surface. A sample of the bacteria causing the infection is taken, usually at the start of the procedure, in order to determine an appropriate antibiotic treatment. Unfortunately, however, bacterial re-infection and biofilm formation is a not-uncommon complication of DAIR surgery. The inventor expect that the present invention may be utilised to debride biofilm during a DAIR procedure, with potentially better outcomes for the patient. The composition of the present invention could, for example, be used to wash out the exposed implant.

In a specific embodiment, a composition in accordance with the present invention might, for example, be administered to the patient using the technique described below.

A composition including a biofilm degrading protease (e.g. bromelain), disulphide bond breaking agent (e.g. acetylcysteine) and, optionally, an additional biofilm degrading agent (e.g. DNase, calcium gluconate, dispersin B and subtilin) and/or an antibiotic (e.g. an IV antibiotic such as gentamicin) may be percutaneously introduced into an infected seroma. For example, 3-5 milligrams of bromelain and 1.4 g of acetylcysteine could be mixed with 70 ml of 5% dextrose and instilled via a percutaneous drain. Any number of repeated doses of identical (or different) formulations may be administered at regular intervals (e.g. every 24 h). The drain may be aspirated before each additional dose in order to clear fluid from around the implant. After the final dose of the bromelain-containing composition is instilled, the drain may be aspirated and 80 mg of gentamicin instilled percutaneously in the same drain. Finally, the drain may be aspirated to dry before its final removal (e.g. on the following day).

### Pharmaceutical Compositions

The combination of biofilm degrading protease, disulphide bond breaking agent and, optionally, additional biofilm degrading agent and/or antibiotic used in the present invention may, in some embodiments, be provided in the form of a pharmaceutical composition comprising a pharmaceutically acceptable carrier.

Such a pharmaceutically acceptable carrier will depend on the route of administration of the composition. Liquid form preparations may include solutions, suspensions and emulsions, for example water or water-propylene glycol solutions for parenteral injection, aerosols or solutions for intranasal or intratracheal delivery. Suitable pharmaceutically acceptable carriers for use in the pharmaceutical compositions of the present invention include physiologically buffered saline, dextrose solutions and Ringer's solution, etc.

Pharmaceutical compositions suitable for delivery to a patient may be prepared immediately before delivery into the patient's body or may be prepared in advance and stored appropriately beforehand.

The pharmaceutical compositions and medicaments for use in the present invention may comprise a pharmaceutically acceptable carrier, adjuvant, excipient and/or diluent. The carriers, diluents, excipients and adjuvants must be “acceptable” in terms of being compatible with the other ingredients of the composition or medicament and the delivery method, and are generally not deleterious to the recipient thereof. Non-limiting examples of pharmaceutically acceptable carriers or diluents which might be suitable for use in some embodiments are demineralised or distilled water; water for injection; saline solution; dextrose solution; vegetable based oils such as peanut oil, safflower oil, olive oil, cottonseed oil, maize oil; sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxylpropylmethylcellulose; lower alkanols, for example ethanol or isopropanol; lower aralkanols; lower polyalkylene glycols or lower alkylene glycols, for example polyethylene glycol, polypropylene glycol, hydrogels, alginate, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from about 10% to about 99.9% by weight of the composition or medicament.

It will be understood that, where appropriate, some of the components in the combination or pharmaceutical compositions may be provided in the form of a metabolite, pharmaceutically acceptable salt, solvate or prodrug thereof.

“Metabolites” of the components of the invention refer to the intermediates and products of metabolism.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Experimental Results

Experiments conducted by the inventor to demonstrate the effect of specific embodiments of the present invention will now be described.

The bromelain used in the experiments described below was manufactured and provided by Mucpharm Pty Ltd (Australia) as a sterile powder. Bromelain was diluted either in phosphate buffered saline (PBS) when used as single agent, or directly in acetylcysteine solution when used as in combination (sometimes referred to as “BromAc” in the examples), to prepare formulations of various concentrations (e.g. 5, 10, 20, 25, 50, 100, 250 and 500 μg/mL). Acetylcysteine 200 mg/ml was purchased from Link Pharma (Australia) and prepared as 5, 10, 20 and 30 mg/ml solutions by dilution in PBS. All other reagents were of Analytical grade from Sigma Aldrich, Sydney, Australia.

### Experiment 1—Effect of Bromelain (75 μg/Ml) and Acetylcysteine on Biofilm Formation of S. aureaus ATCC6538

S. aureus ATCC 6538 were cultured in L-broth at 37° C. for 24 hrs, after which 200 uL of bacterial culture was added to each well of a 24 well plate. Each plate was treated at the same time with 75 μg/ml bromelain and 75 μg/ml bromelain with different concentration of acetylcysteine (1%, 0.5% and 0.25%). The plate was then incubated at 37° C. for 48 hrs.

After this time, the plate was washed and stained with 0.1% crystal violet and the amount of crystal violet binding was measured by destaining the biofilms for 5 min with 400 u1 of ethanol. The optical density of the biofilm was measured at 570 nm.

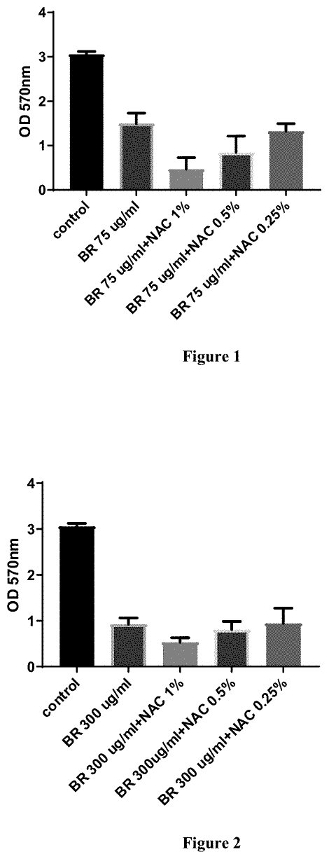

The results are shown in FIG. 1 and demonstrate a synergistic effect when relatively low amounts of bromelain and acetylcysteine are used in combination.

### Experiment 2—Effect of Bromelain (300 μg/Ml) and Acetylcysteine on Biofilm Formation of S. aureaus ATCC6538

S. aureus ATCC 6538 were cultured in L-broth at 37° C. for 24 hrs, after which 200 uL of bacterial culture was added to each well of a 24 well plate. Each plate was then treated at the same time with 300 μg/ml bromelain and 300 μg/ml bromelain with different concentration of acetylcysteine (1%, 0.5% and 0.25%). The plate was then incubated at 37° C. for 48 hrs.

After this time, the plate was washed and stained with 0.1% crystal violet and the amount of crystal violet binding was measured by destaining the biofilms for 5 min with 400u1 of ethanol. The optical density of the biofilm was measured at 570 nm.

The results are shown in FIG. 2 and demonstrate a synergistic effect, even when relatively high amounts of bromelain are used in combination with acetylcysteine. Some patients may be able to tolerate amounts of bromelain as high as this.

### Experiment 3—Anti-Biofilm Activity of Bromelain, Acetylcysteine and, Optionally, Gentamicin on 24-Hour Staphylococcus aureus ATCC 6538 Biofilms

S. aureus ATCC 6538 were cultured in L-broth at 37° C. for 24 hrs or 48 hrs to form a biofilm, after which 200 uL of bacterial culture was added to each well of a 24 well plate. Each plate was then incubated at 37° C. for 24 hrs and, afterwards, the plates were treated with 20 mg/mL (2%) acetylcysteine, various concentrations of bromelain, gentamicin and combinations thereof. The plates were then incubated at 37° C. for 4 hrs.

After treatment, the cells were fixed with 99% methanol and stained with 1% crystal violet. The stained biofilm was dissolved with 33% acetic acid and the absorbance was read at 590 nm. The results were analyzed using Prism GraphPad software.

### Experiment 3.1—4-Hour Treatment with Bromelain (5 μg/ml-100 μg/Ml) and Acetylcysteine (20 mg/mL) on 24-hour Staphylococcus aureus ATCC 6538 Biofilms

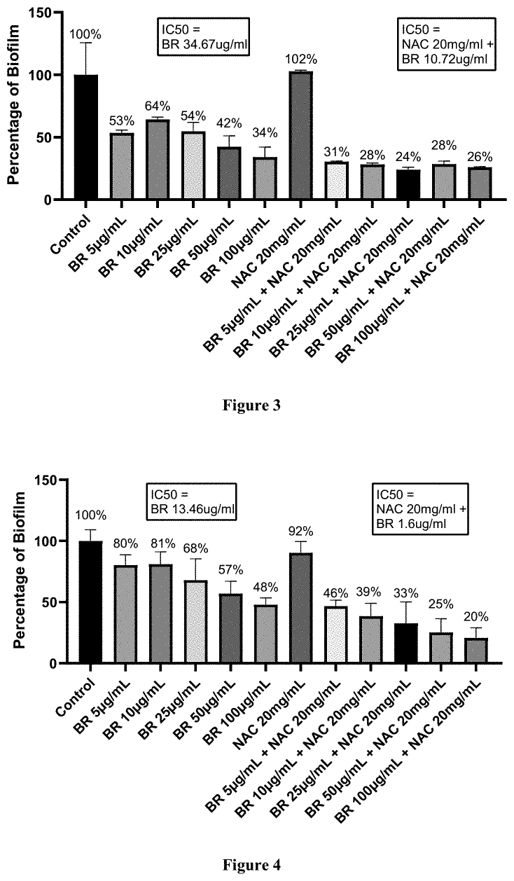

The results of this experiment are shown in FIG. 3 and demonstrate that the most effective single treatment bromelain was 100 μg/ml, with 66% of the biofilm eradicated. The effectiveness of biofilm eradication for bromelain as a single agent was reduced with a decrease in concentration by approximately 10% of biofilm for each concentration decrease apart from 5 ug/ml. The single treatment of 20 mg/mL acetylcysteine had no effect on biofilm eradication and resulted in a higher percentage of biofilm compared to the control. The combination treatments of bromelain and 20 mg/ml acetylcysteine resulted in >70% biofilm eradication, with the most effective treatment being the 25 ug/mL Bromelain+20 mg/mL acetylcysteine which reduced 76% of preformed biofilms. As can be seen, however, each bromelain+acetylcysteine treatment was more effective at biofilm eradication compared to bromelain or acetylcysteine as single agents.

### Experiment 3.2—4-Hour Treatment with Bromelain (5 μg/ml-100 μg/ml) and Acetylcysteine (20 mg/mL) on 48-Hour Staphylococcus aureus ATCC 6538 Biofilms

The results of this experiment are shown in FIG. 4. In agreement with previous results, the most effective single treatment bromelain was 100 m/ml, however, the eradication of biofilm was only 52%. When compared to the 24-hour biofilm treatment (Experiment 3.1), which at this concentration eradicated 66% of the biofilm, we can see a correlation between a decrease in effectiveness of treatment and time of preformed biofilm. The effectiveness of bromelain as a single agent at eradicating preformed biofilms was reduced by with each decrease in concentration with the exception of the concentration 5 μg/ml, which appears to plateau.

In line with previous results, the combination treatments of 20 mg/ml acetylcysteine and bromelain resulted in a greater effectiveness at biofilm eradication when compared to the single agent treatments. All combination treatments resulted in >53% of biofilm eradication. For the 48-hour biofilm, the most effective treatment was the 20 mg/mL acetylcysteine+100 μg/mL Bromelain, which reduced 79.3% of preformed biofilms. However, each bromelain+acetylcysteine treatment again had a reduction in effectiveness with each decrease in concentration.

The experiments described above demonstrate that the combination of bromelain and acetylcysteine show a clear synergy against biofilms.

### Experiment 3.3—4-Hour Treatment with Bromelain (25 μg/mL), Acetylcysteine (20 mg/mL) and Gentamicin (12 μg/ml, 16 μg/ml and 35 μg/ml) on 24-Hour Staphylococcus aureus ATCC 6538 Biofilms

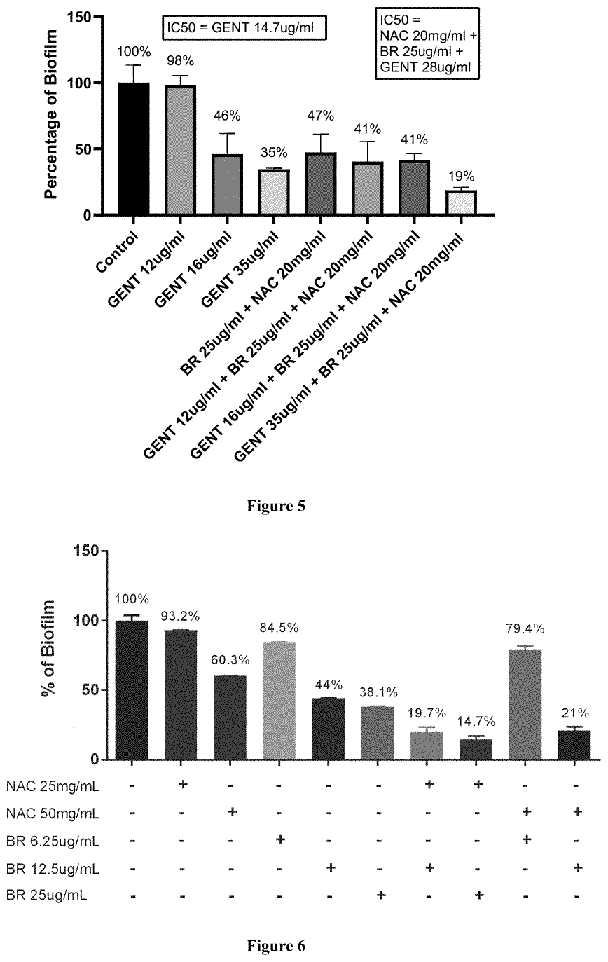

The results of these experiments are shown in FIG. 5. Clear synergy can be seen with the lower gentamicin doses, with the 12 μg/ml gentamicin treatment resulting in 2% eradication, but when combined with bromelain+acetylcysteine the eradication increases to 59%. The 16 μg/ml gentamicin as a single agent was only effective by 5% compared to the concentration in combination. The highest examined concentration of Gentamicin (35 μg/ml) also showed clear synergy, with an additional 15% eradication observed in the combination treatment. As expected, the best overall treatment was the gentamicin 35 μg/ml+bromelain 25 μg/ml+acetylcysteine 20 mg/ml.

The most effective gentamicin treatment was 35 μg/ml with approximately 60% eradication (and an IC50 of 14.13 m/m1). The combination of gentamicin, bromelain, and acetylcysteine showed clear synergy, primarily with the lower gentamicin doses. Interestingly, the bromelain and acetylcysteine combination treatments are as effective as the antibiotics as single agents.

### Experiment 4—Anti-Biofilm Activity of Bromelain, Acetylcysteine and, Optionally, Ampicillin on 48-Hour Pseudomonas aeruginosa ATCC 31461 Biofilms

Pseudomonas aeruginosa ATCC 31461 were cultured in Trypsin Soy broth at 37° C. for 48 hrs to form a biofilm, after which 200 uL of bacterial culture was added to each well of a 24 well plate. Each plate was then incubated at 37° C. for 24 hrs and, afterwards, the plates were treated with various concentrations of acetylcysteine, bromelain and/or ampicillin. The plate was then incubated at 37° C. for 4 hrs.

After treatment, the cells were fixed with 99% methanol and stained with 1% crystal violet. The stained biofilm was dissolved with 33% acetic acid and the absorbance was read at 590 nm. The results were analyzed using Prism GraphPad software.

### Experiment 4.1—4-Hour Treatment with Bromelain and Acetylcysteine on 48-Hour Pseudomonas aeruginosa ATCC 31461 Biofilms

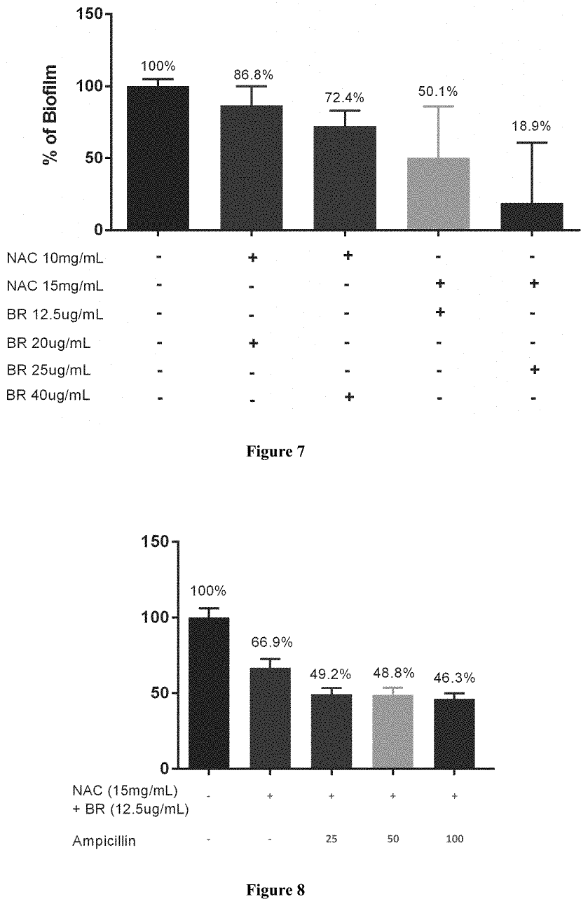

The results of these experiments are shown in FIGS. 6 and 7. Referring firstly to FIG. 6, it can be seen that acetylcysteine as a single agent is not effective at 25 mg/mL but is moderately effective at 50 mg/ml. Bromelain as a single agent had no substantial effect at 6.25 μg/mL but eradicated approximately 56% of preformed biofilm with the 12.5 μg/mL and 62% for the 25 μg/mL treatment. The combination of 50 mg/mL NAC+6.5 μg/mL Bromelain had minimal eradication effect, however, when compared to the other combination treatments, there is obvious error with this reading. The remaining combination treatments resulted in over 79% of biofilm eradication. The most effective combination was the 25 mg/mL NAC+25 μg/mL Bromelain, with 85% of the preformed biofilm eradicated.

Referring now to FIG. 7, it can be seen that the combinations with acetylcysteine concentrations at 10 mg/mL were not substantially effective at eradicating preformed biofilms when combined with 20 μg/mL and 40 μg/mL of Bromelain. Increasing the acetylcysteine concentration to 15 mg/mL, however, resulted in over 50% of biofilm eradication for bromelain 12.5 μg/mL and over 81% for Bromelain 25 μg/mL. The most effective combination was the 15 mg/mL NAC+25 μg/mL Bromelain, with over 81% of the preformed biofilm eradicated.

### Experiment 4.2—4-Hour Treatment with Bromelain, Acetylcysteine and Ampicillin on 48-Hour Pseudomonas aeruginosa ATCC 31461 Biofilms

The results of this experiment are shown in FIG. 8. As previously observed, the combination of bromelain and acetylcysteine eradicated approximately 30% of the preformed biofilm. The addition of ampicillin was shown to marginally increase the combination's effectiveness at all examined concentrations, albeit by about 20% for the examined concentrations. No significant difference was observed and, due to this, further experiments will focus on a wider set of concentrations in combination with bromelain and acetylcysteine.

### Experiment 5—Anti-Biofilm Activity of Bromelain, Acetylcysteine and, Optionally, Gentamicin or Ampicillin on 48-Hour Pseudomonas aeruginosa PA01 398 Biofilms

Pseudomonas aeruginosa PA01 398 were cultured in Trypsin Soy broth at 37° C. for 48 hrs to form a biofilm, after which 200 uL of bacterial culture was added to each well of a 24 well plate. Each plate was then incubated at 37° C. for 24 hrs and, afterwards, the plates were treated with various concentrations of acetylcysteine, bromelain, gentamicin, ampicillin and combinations thereof. The plate was then incubated at 37° C. for 4 hrs.

After treatment, the cells were fixed with 99% methanol and stained with 1% crystal violet. The stained biofilm was dissolved with 33% acetic acid and the absorbance was read at 590 nm. The results were analyzed using Prism GraphPad software.

### Experiment 5.1—4-Hour Treatment with Bromelain and Acetylcysteine on 48-Hour Pseudomonas aeruginosa PA01 398 Biofilms

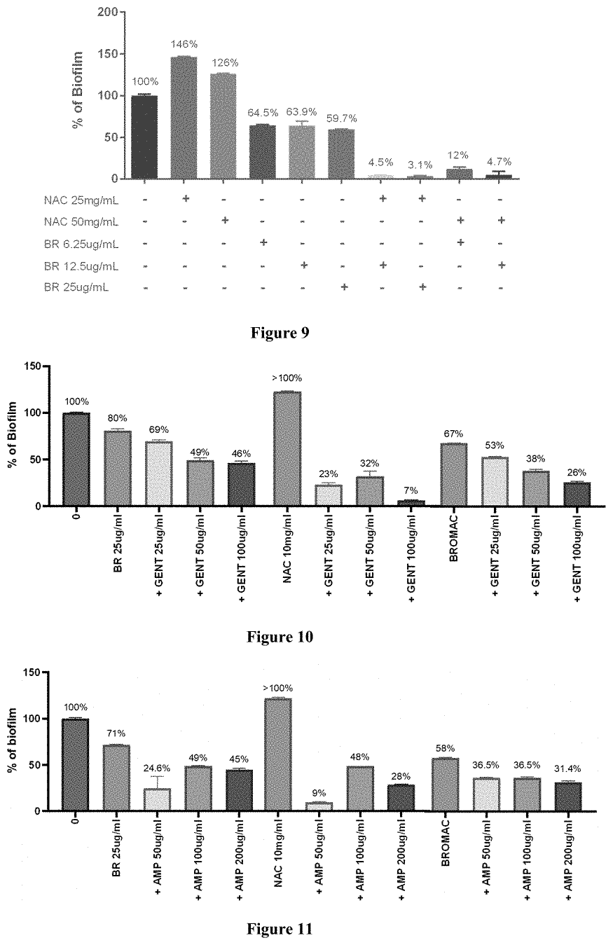

In the first experiment, the results of which are presented in FIG. 9, it can be seen that NAC as a single agent is antagonistic in both concentrations examined. Bromelain as a single agent eradicates approximately 35% of preformed biofilm with minimal variation (<5%) between the three examined concentrations. All combinations of bromelain and acetylcysteine, however, showed a significant increase in biofilm eradication, with all treatments resulting in over 88% eradication. The most effective combination was the 25 mg/mL NAC+25 μg/mL Bromelain with 96.9% of the preformed biofilm eradicated.

### Experiment 5.2—4-Hour Treatment with Bromelain, Acetylcysteine and Gentamicin on 48-Hour Pseudomonas aeruginosa PA01 398 Biofilms

The results of these experiments are shown in FIG. 10. As previously the combination of bromelain and acetylcysteine was observed to be more effective than either agent alone. All combination treatments show synergy, with a clear reduction in biofilm with the addition of gentamicin to the bromelain/acetylcysteine combination.

### Experiment 5.3—4-Hour Treatment with Bromelain, Acetylcysteine and Ampicillin on 48-Hour Pseudomonas aeruginosa PA01 398 Biofilms

The results of these experiments are shown in FIG. 11. As previously, the combination of bromelain and acetylcysteine was observed to be more effective than either agent alone. All combination treatments show synergy with a clear reduction in biofilm with the addition of ampicillin.

### Experiment 6—Comparison of Combination Treatments Versus Staged Treatments on 48-Hour Pseudomonas aeruginosa PA01 398 Biofilms

Pseudomonas aeruginosa PA01 398 were cultured in Trypsin Soy broth at 37° C. for 48 hrs to form a biofilm, after which 200 uL of bacterial culture was added to each well of a 24 well plate. Each plate was then incubated at 37° C. for 24 hrs and, afterwards, the plates were treated with various concentrations of acetylcysteine, bromelain and either gentamicin or ampicillin. The plate was then incubated at 37° C. for 4 hrs. After treatment, the cells were fixed with 99% methanol and stained with 1% crystal violet. The stained biofilm was dissolved with 33% acetic acid and the absorbance was read at 590 nm. The results were analyzed using Prism GraphPad software.

### Experiment 6.1—Combination Treatment Including Ampicillin

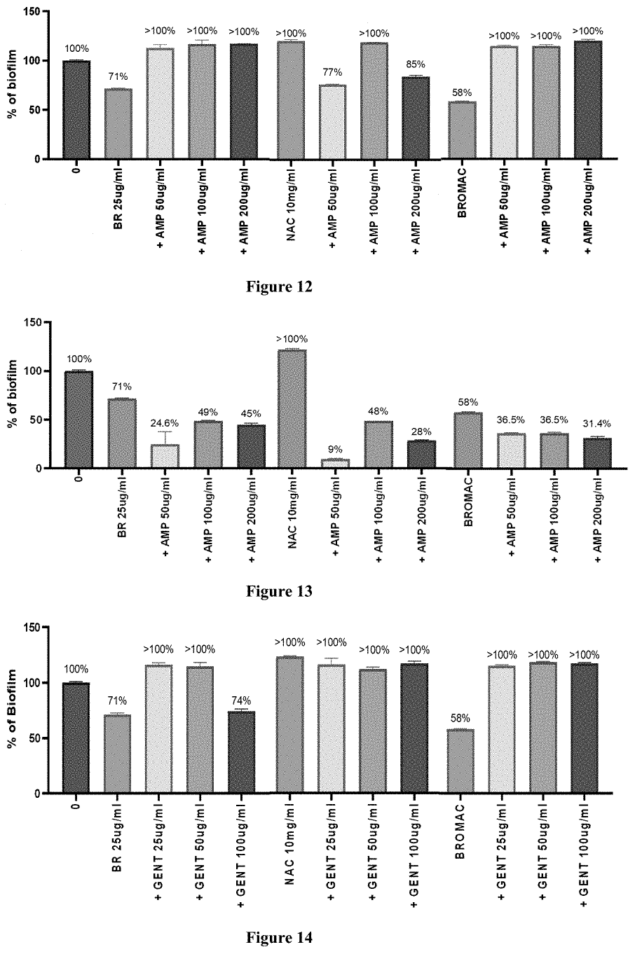

In the first series of experiments, the results of which are shown in FIG. 12, 48-hour Pseudomonas aeruginosa PA01 398 biofilms were treated for 4-hours with the compositions shown in the table including ampicillin (50-200 μg/mL), bromelain (25 μg/mL) and acetylcysteine (10 mg/mL) in 5% glucose.

As can be seen, the combination of bromelain and acetylcysteine (“BROMAC” in the Figure) is more effective than either agent alone. All combination treatments show antagonism when the components are used together.

### Experiment 6.2—Staged Treatment Including Ampicillin

In the next series of experiments, the results of which are shown in FIG. 13, 48-hour Pseudomonas aeruginosa PA01 398 biofilms were treated for 4-hours with bromelain (25 μg/mL), acetylcysteine (10 mg/mL) or a combination of the two (“BROMAC”) in 5% glucose. Subsequently, the biofilm was washed and was then treated with ampicillin (50-200 μg/mL) for 4 hours.

As can be seen, all combination treatments showed synergy, with a clear reduction in biofilm with the addition of ampicillin. Further concentrations of bromelain and acetylcysteine need to be examined in addition to ampicillin.

### Experiment 6.3—Combination Treatment Including Gentamicin

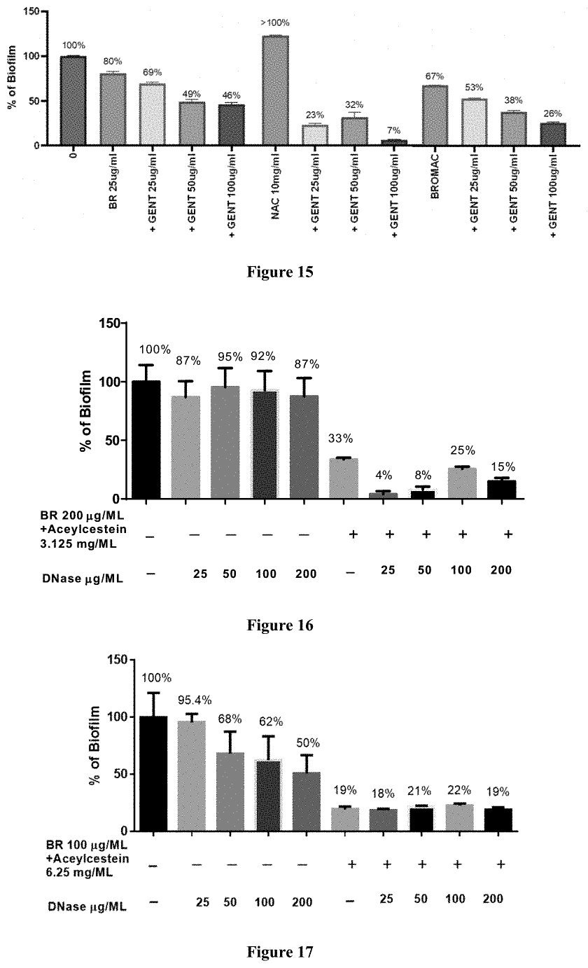

In the third series of experiments, the results of which are shown in FIG. 15, 48-hour Pseudomonas aeruginosa PA01 398 biofilms were treated for 4-hours with the compositions shown in the table including Gentamicin (20-100 μg/mL), bromelain (25 μg/mL) and acetylcysteine (10 mg/mL) in 5% glucose.

As can be seen, the combination of bromelain and acetylcysteine (“BROMAC” in the Figure) is more effective than either agent alone. All combination treatments show antagonism when the components are used together.

### Experiment 6.4—Staged Treatment Including Gentamicin

In the final series of experiments, the results of which are shown in FIG. 16, 48-hour Pseudomonas aeruginosa PA01 398 biofilms were treated for 4-hours with bromelain (25 μg/mL), acetylcysteine (10 mg/mL) or a combination of the two (“BROMAC”) in 5% glucose. Subsequently, the biofilm was washed and was then treated with Gentamicin (25-100 ng/mL) for 4 hours.

As can be seen, all combination treatments showed synergy, with a clear reduction in biofilm with the addition of gentamicin. Further concentrations of bromelain and acetylcysteine need to be examined in addition to gentamicin.

### Experiment 7—Eradication Effect of the Bromelain/Acetylcysteine Combination with DNase Against Staphylococcus aureus ATTC 6538 Biofilm

In these experiments, the eradication effect of combinations of bromelain and acetylcysteine with DNase were tested against a Staphylococcus aureus ATTC 6538 biofilm over a 4 hour treatment.

S. aureus 6538 culture (OD.37) were plated in (400 uL) 24 well plates and allowed to grow for 48 hrs. After incubation, the supernatant was carefully removed with a transfer pipette and each well was washed twice with ice cold PBS buffer. The plates were then treated for 4 hrs with DNase (at concentrations of 25, 50, 100 and 200 μg/ml), with or without either 100 or 200 μg/ml bromelain and 6.25 or 3.125 mg/ml acetylcysteine, respectively.

After 4 hours, the plates were washed with PBS and fixed with methanol for 15 minutes, after which the methanol was discarded and the plates were washed twice with PBS. The plates were then stained for 10 minutes with 0.1% crystal violet, after which the crystal violet was discarded and plates were again washed twice with PBS. Plates were left in a hood for drying for 2 hrs, after which 400 μl of 33% acetic acid was added in each well (10 minutes) to dissolve the crystal violet. Finally, the optical density of the biofilm at 590 nm was measured.

The results of these experiments are shown in FIG. 16 (200 μg/ml bromelain and 3.125 mg/ml acetylcysteine) and FIG. 17 (100 μg/ml bromelain and 6.25 mg/ml acetylcysteine), and show that combinations of bromelain/acetylcysteine and DNase are more effective at reducing biofilm than just DNase.

As described herein, the present invention provides compositions and methods for debriding biofilm on implants in a patient's body. Embodiments of the present invention provide a number of advantages over existing therapies, some of which are described above.

It will be understood to persons skilled in the art of the invention that many modifications may be made without departing from the spirit and scope of the invention. All such modifications are intended to fall within the scope of the following claims.

It will be also understood that while the preceding description refers to specific forms of the microspheres, pharmaceutical compositions and methods of treatment, such detail is provided for illustrative purposes only and is not intended to limit the scope of the present invention in any way.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A composition comprising a biofilm degrading protease, a disulphide bond breaking agent and an antibiotic.

2. The composition of claim 1, wherein the biofilm degrading protease is a cysteine protease.

3. The composition of claim 1, wherein the biofilm degrading protease is selected from one or more of the group consisting of: bromelain, papain, ficain, actinidain, zingibain, fastuosain and ananain.

4. The composition of claim 1, wherein the disulphide bond breaking agent is acetylcysteine.

5. The composition of claim 1, wherein the antibiotic is selected from one or more of the group consisting of: an aminoglycoside, a cephalosporin antibiotic and a penicillin antibiotic.

6. The composition of claim 1, further comprising an additional biofilm degrading agent which degrades one or both of the DNA or PNAG components of biofilm.

7. (canceled)

8. The composition of claim 6, wherein the biofilm degrading agent is selected from one or more of the group consisting of: DNase, calcium gluconate, dispersin B and subtilin.

9. The composition of claim 1, wherein the composition is adapted for injection into a patient or for inhalation by a patient.

10. (canceled)

11. A method for debriding biofilm on an implant in a patient's body, the method comprising contacting the biofilm with a combination of a biofilm degrading protease and a disulphide bond breaking agent.

12. The method of claim 11, wherein the biofilm degrading protease and disulphide bond breaking agent are administered simultaneously or sequentially.

13. The method of claim 11, wherein contacting the biofilm with the combination of the biofilm degrading protease and disulphide bond breaking agent comprises injecting the combination proximal to the implant.

14. The method of claim 11, further comprising aspirating a resultant fluid from proximal to the implant.

15. (canceled)

16. The method of claim 11, wherein the biofilm degrading protease is a cysteine protease selected from one or more of the group consisting of: bromelain, papain, ficain, actinidain, zingibain, fastuosain and ananain.

17. (canceled)

18. The method of claim 11, wherein the disulphide bond breaking agent is acetylcysteine.

19. The method of claim 11, further comprising contacting the biofilm with an antibiotic.

20. The method of claim 19, wherein the antibiotic is administered simultaneously with or sequentially to the biofilm degrading protease and/or disulphide bond breaking agent, or the antibiotic is administered after the biofilm has been debrided.

21-22. (canceled)

23. A method for treating a bacterial infection involving biofilm in a patient, the method comprising administering to the patient a combination of a biofilm degrading protease, a disulphide bond breaking agent and an antibiotic.

24. The method of claim 23, wherein the combination is administered to the patient by injection or by inhalation.

25. The method of claim 24, wherein the bacterial infection involving biofilm is a secondary bacterial infection as a result of cystic fibrosis, COPD, severe asthma or other respiratory disease.

26. A method for treating a bacterial infection on an implant in a patient's body, the method comprising contacting the implant with a combination of a biofilm degrading protease, a disulphide bond breaking agent and an antibiotic.

27-38. (canceled)
